(19) United States
(12) Patent Application Publication
(10) Pub. No.: US 2016/0000588 A1
(43) Pub. Date: Jan. 7, 2016

(54) SELF-EXPANDING BIFURCATED STENT

(71) Applicant: RUSH UNIVERSITY MEDICAL CENTER, Chicago, IL (US)

(72) Inventor: Andrew K. JOHNSON, Chicago, IL (US)

(21) Appl. No.: 14/769,878

(22) PCT Filed: Feb. 18, 2014
(86) PCT No.: PCT/US14/16917

§ 371 Date: Aug. 24, 2015

Related U.S. Application Data
(60) Provisional application No. 61/778,040, filed on Mar. 12, 2013.

(51) Int. Cl. A61F 2/856; A61F 2/97; A61F 2/90; A61F 2/844

(52) U.S. Cl. CPC A61F 2/856; A61F 2/844; A61F 2/97; A61F 2/90; A61F 2002/828

(57) ABSTRACT

A self-expanding stent is disclosed, the self-expanding stent having a collapsed configuration and an expanded configuration. The self-expanding stent has three stent subunits, each including one or more longitudinally-oriented diamonds and one or more axially-oriented diamonds. The subunits are coupled together at a pivot joint. The longitudinally-oriented diamonds are configured to substantially hold their shape in both the expanded configuration of the stent and the collapsed configuration of the stent. The axially-oriented diamonds are configured to expand from the collapsed configuration to the expanded configuration. Expansion of the axially-oriented diamonds creates angulation between the stent subunits at the pivot joint. Thus, the stent can be inserted into a target location through a tubular catheter, once the stent is no longer constrained by the catheter, it will expand to an angled configuration.

This application claims the benefit of priority under 35 U.S.C. §119(e) to U.S. Provisional Application No. 61/778,040, filed Mar. 12, 2013. The disclosures set forth in the referenced application is incorporated herein by reference in its entirety.

### BACKGROUND

Stents can be placed within the mammalian vasculature using endovascular techniques for the treatment of diseased vessels. Applications of stents include treatment of stenotic and atherosclerotic lesions in the coronary, peripheral, and cerebral vasculature. Another common application of stents is the treatment of cerebral aneurysms. Stents are designed to oppose the subject's inner vascular walls and provide an unobstructed conduit for blood flow within the stent lumen.

Stents are generally designed as straight homogenous tubes using biocompatible materials designed to treat the vessel pathology. Placing stents into vessel bifurcations requires deployment of multiple stents given the materials currently available. When more than one device is placed with overlap, the risk of complication exponentially increases—vessel wall apposition is decreased and stent material of an overlapped stent extends into the vessel lumen more than stent material of a single, non-overlapping stent would extend into the vessel.

There are several stent designs to overcome the problems associated with a bifurcation, but no current design is completely satisfactory for all applications. Highly flexible stents have been designed to fit the curvature of a bifurcated vessel but are not capable of extending through multiple branched vessels. Flexible stents with expanding elements may extend through bifurcated vessels slightly more, yet still incompletely. Other bifurcation reconstruction devices offer a solution for bifurcations but do not allow customization.

### SUMMARY

In illustrative embodiments, a self-expanding stent is disclosed, the self-expanding stent having a collapsed configuration and an expanded configuration. The self-expanding stent includes three stent subunits, which each include one or more longitudinally-oriented diamonds and one or more axially-oriented diamonds, as more fully described below. In an illustrative embodiment, the three subunits are oriented in the same plane and their terminal longitudinal diamonds are coupled together by a connector at a pivot joint. The terminal axial diamonds of the first subunit are also connected to axial subunits of the second and third stent subunits. The longitudinally-oriented diamonds are configured to substantially hold their shape in both the expanded configuration of the stent and the collapsed configuration of the stent. The axially-oriented diamonds are configured to expand from the collapsed configuration to the expanded configuration. The design allows the angle between the first stent subunit and the second and third stent subunits to enlarge with stent crimping and lessen with stent expansion. Thus, the stent can be inserted into a target location through a tubular catheter, and after the stent is no longer constrained by the catheter, it will expand to an angled configuration.

In another illustrative embodiment, the three subunits and connectors are replaced by a single component incorporating the shape and function described above. This can be accomplished by three-dimensional printing of a shape-memory material. This method allows for full customization of the stent, with respect to 1) stent angulation with expansion, 2) regional porosity, 3) regional stent radial force, 4) length of each subunit, 5) regional drug-elution, and 6) additional unforeseen customization requirements.

### BRIEF DESCRIPTION OF THE DRAWINGS

FIG. 1A is a top elevation view of an unrolled and expanded subunit of a stent in accordance with the present disclosure;

FIG. 1B is a top elevation view of the stent of FIG. 1A in a rolled and collapsed state;

FIG. 2A is a top elevation view of a first stent component of a stent in accordance with the present disclosure, the first stent component in an unrolled and expanded state;

FIG. 2B is a side view of the first stent component of FIG. 2A in a rolled and expanded state;

FIG. 2C is a front view of the first stent component of FIG. 2A in a rolled and expanded state;

FIG. 3A is a top elevation view of a second stent component of a stent in accordance with the present disclosure, the second stent component in an unrolled and expanded state;

FIG. 3B is a bottom view of the second stent component of FIG. 3A in a rolled and expanded state;

FIG. 3C is a side view of the second stent component of FIG. 3A in a rolled and expanded state;

FIG. 4A is a top elevation view of a branched wire of the present disclosure;

FIG. 4B is a top elevation view of a stent of the present disclosure in a rolled and collapsed stated;

FIG. 4C is a top elevation view of the branched wire of FIG. 4A inside the rolled and collapsed stent of FIG. 4B;

FIG. 5 is a side perspective view of a stent of the present disclosure in a rolled and partially expanded state;

FIG. 6 is a side perspective view of the stent of FIG. 5 in a rolled and fully expanded state;

FIG. 7A is an isometric view of a stent of the present disclosure in a rolled and collapsed state;

FIG. 7B is an enlarged isometric view of a portion of FIG. 7A, wherein the enlarged view depicts a junction in which arms of a second stent component are attached to a first stent component; and

FIG. 8 is a top isometric view of the stent of FIGS. 7A and 7B in a rolled and fully expanded state.

### DETAILED DESCRIPTION

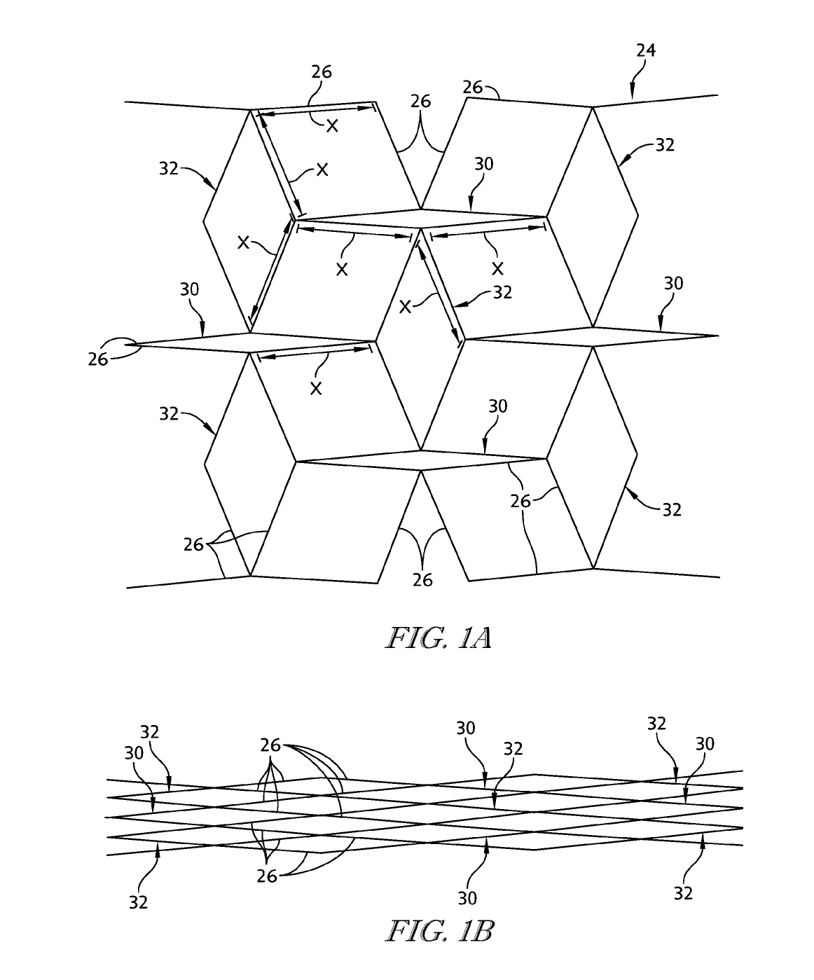

An illustrated stent 10 in which the principles of the present disclosure may be implemented includes a first stent component 14, a second stent component 12 that is bifurcated or branched, and one or more junction points 16 configured to join the first second component 14 to the second stent component 12 at a junctional region 38. The first stent component 14 and second stent component 12 are configured to include one or more longitudinally-oriented diamonds 30 and one or more axially-oriented diamonds 32 that, in part, form the structure of the stent 10. As illustrated in FIGS. 1A and 1B, the longitudinally-oriented diamonds 30 are configured to change shape less than the axially-oriented diamonds 32. The axially-oriented diamonds 32 are configured to be movable from a first shape similar to the longitudinally-oriented diamonds 30 in a collapsed state to a second shape that is wider in the expanded state. The designs of the diamonds 30 and 32 allow the angle between the first stent component 14 and the second stent component 12 to enlarge with stent crimping and lessen with stent expansion.

To form the stent 10, the first stent component 14 is coupled to the second stent component 12 at apices 42 of the longitudinally-oriented diamonds 30. When the stent 10 is in a collapsed state, the stent 10 may be inserted into a patient's blood vessel, for instance by a catheter (not shown), during angioplasty or other types of medical procedures and positioned to expand in the vessel at a point where the vessel branches or bifurcates into two or more vessels. The use of such catheter or insertion techniques may be as described in pending application number WO 2013/009976, the contents of which are incorporated herein by reference.

The first stent component 14 and the second stent component 12 of the stent 10 are self-expanding elements. The first stent component 14 is configured to be placed in the proximal lumen of a vessel prior to a bifurcation; the second stent component 12 is configured to be placed in the lumens of both distal vessels of a bifurcation (after the bifurcation occurs). The second stent component 12 can be produced as two joined subunits 24 or as a single piece. The two stent components 14, 12 will be connected at the junction point 16 at apices 42, 44 of the longitudinally-oriented and axially-oriented diamonds 30, 32, respectively, so that the junctional region 38 is aligned with a bifurcating point of the blood vessels (not shown) after the stent 10 is deployed in an expanded state. In an illustrative embodiment, the first stent component 14 and the second stent component 12 are laser-cut from Nitinol tubing, but other materials and methods are envisioned.

FIGS. 1A and 1B illustrate an unrolled geometry of a stent subunit 24 of the present disclosure. Specifically, FIG. 1A depicts an open or expanded configuration of an unrolled stent subunit 24, and FIG. 1B depicts a closed or collapsed configuration of an unrolled stent subunit 24. The stent subunit 24 is configured to be coupled together with one or more other stent subunits 24 in order to form the first stent component 14 or the second stent component 12. A portion of the stent subunit 24 may also be used to achieve the same effect. As illustrated, the stent subunit 24 includes the longitudinally-oriented diamonds 30 and the axially-oriented diamonds 32, each of which are formed by four struts 26 of a predetermined length X. The longitudinally-oriented diamonds 30 tend to hold their shape in both the expanded configuration and the collapsed configuration, as illustrated in FIGS. 1A and 1B. Unlike the longitudinally-oriented diamonds 30, the axially-oriented diamonds 32 are configured to be spread wide in the open/expanded configuration but assume the shape of the longitudinally-oriented diamonds 30 in the closed/collapsed configuration. The axially-oriented diamonds 32 from one stent subunit 24 are configured to be connected to matching axially-oriented diamonds 32 from another stent subunit 24. In this way, the subunits 24 may be parallel in the closed/collapsed configuration. Due to longitudinal compression forces on the diamonds 30, 32 in the open/expanded configuration, however, a connection between multiple subunits 24 may force a bend between the subunits 24 in the open/expanded configuration.

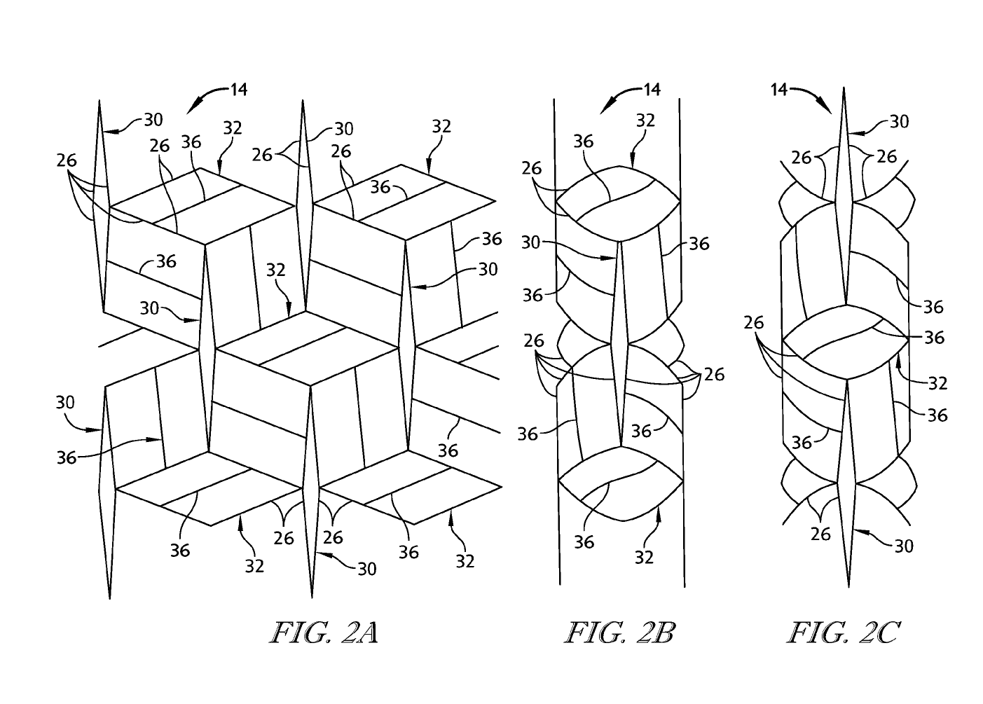

FIGS. 2A, 2B and 2C show an illustrative embodiment of the first stent component 14 as formed by combining one or more subunits 24 from FIGS. 1A and 1B. The first stent component 14 may be cut from a nitinol tube. Specifically, FIG. 2A illustrates an expanded configuration of an unrolled first stent component 14 and demonstrates the shape of circumferential laser-cutting that may be used to form the first stent component 14. FIG. 2B and 2C illustrate the side and front views, respectively, of an expanded configuration of the first stent component 14 in a rolled state and demonstrate illustrative dimensions of the first stent component 14. The longitudinally-oriented diamonds 30 and the axially-oriented diamonds 32, and the struts 26 forming the same, are depicted similar to those in FIG. 1A and 1B. As illustrated in FIG. 2B, additional median struts 36 of the same length X may also be added to bisect the axially-oriented diamond 32. These median struts 36 or other additional struts may collapse completely in the closed form while providing additional strength, radial force, and stability to the stent 10.

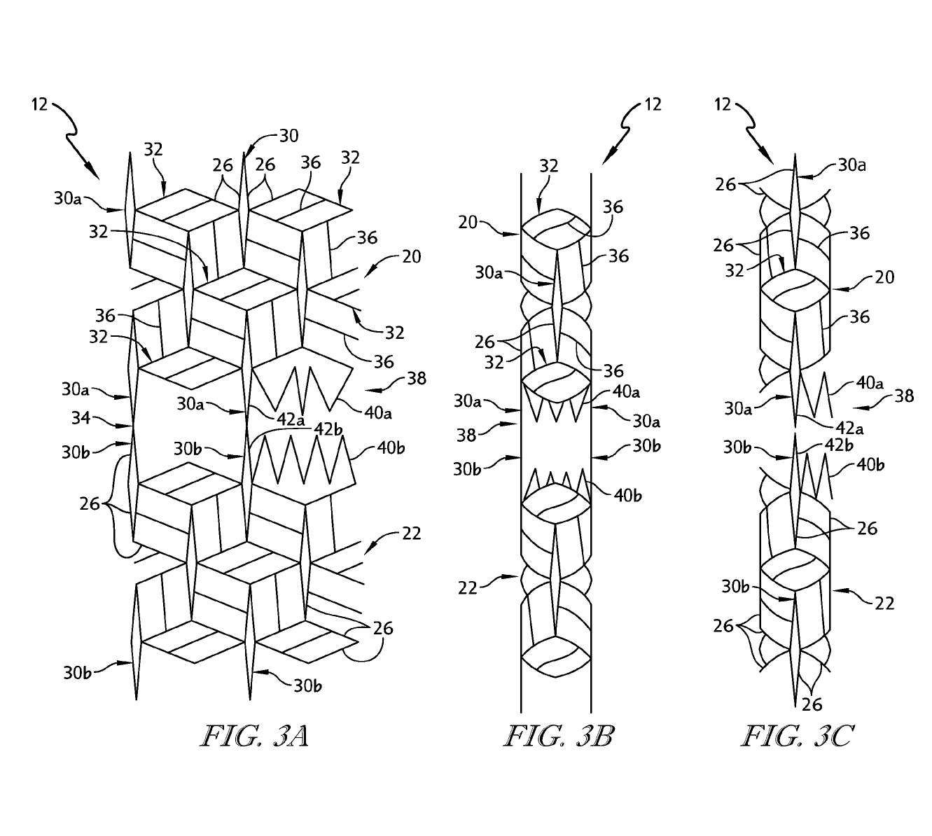

FIGS. 3A, 3B and 3C shows an illustrative embodiment of the second stent component 12, which may also be cut from a single nitinol tube and formed from combining multiple subunits 24. Specifically, FIG. 3A illustrates an expanded configuration of an unrolled second stent component 12, and FIGS. 3B and 3C illustrate the bottom and side views, respectively, of an expanded configuration of the second stent component 12 in a rolled state. The second stent component 12 includes two symmetric or asymmetric arms 20, 22 which connect at a pivot joint 34. The longitudinally-oriented diamonds 30, the axially-oriented diamonds 32, the struts 26 forming the same and the median struts 36 are consistent with the function and geometry of those components in FIGS. 1A-2C. While the illustrated embodiment of the stent 10 comprises a second stent component 12 that includes two symmetric or asymmetric arms 20, 22, the design allows precision orientation even if one of the two arms 20, 22 are omitted.

In illustrative embodiments, a longitudinally-oriented diamond 30a from a first subunit 24 is coupled to a longitudinally-oriented diamond 30b from a second subunit 24 at the apex or apices 42a and 42b of the longitudinally-oriented diamonds 30a and 30b, respectively. Therefore, pivot joint 34 is formed by connecting the apices 42a and 42b and may be cut from the same nitinol tube as both arms 20, 22 or the longitudinally-oriented diamonds 30a and 30b. In alternative embodiments, the longitudinally-oriented diamonds 30a and 30b or their apices 42a and 42b may be altered to allow a separate nitinol, other metal (including platinum or other radiopaque metal), or other biocompatible joining mechanism to be used to form the pivot joint 34.

The bottom of the second stent component 12 may be open so that the lumen of the second stent component 12 can communicate or be joined with another component or subunit 24, including but not limited to the first stent component 14. This connection may be configured to be located at or near the pivot joint 34. The top of the second stent component 12 may include a junctional region 38 that allows additional stent coverage with minimal loss of flexibility the pivot joint 34 of the stent 10. In one embodiment, the junctional region 38 may include first and second scaffolding 40a and 40b, as illustrated in FIG. 3A, that are fitted to the second stent component 12 for such a purpose. In another embodiment, a flexible and collapsible nitinol design may be extend in the junctional region 38 to provide flexibility of other variations of the first and second scaffolding 40a and 40b. In yet another embodiment, the scaffolding 40a and 40b may be connected by separate metal or biocompatible flexible and/or elastic joining material. The opposing scaffolding 40a and 40b of the junctional region 38 can be varied so that this portion of the stent 10 can be tailored to the desired anatomy/pathology.

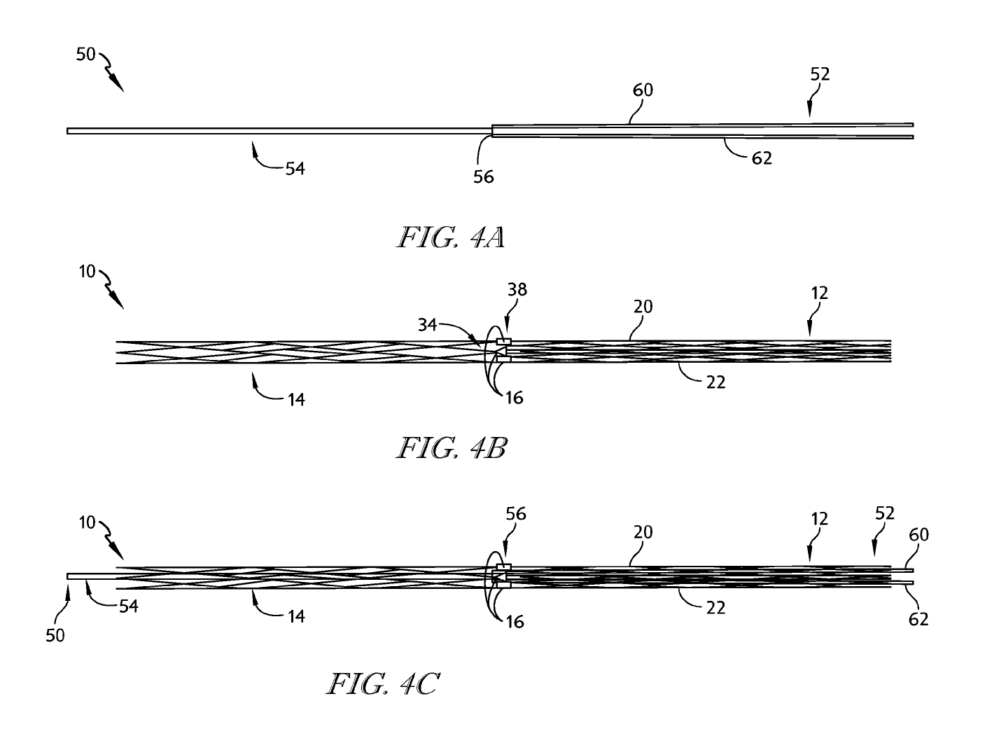

FIG. 4A shows a branched wire 50 that may be used to assist with placement and expansion of the stent 10 inside a blood vessel. Specifically, the branched wire 50 includes a proximal end 54, a distal end 52, and a junction 56 that couples the proximal end 54 to the distal end 52. The distal end 52 includes two arms 60 and 62 joined together at the junction 56. In general, when the stent 10 is in a collapsed state, the proximal end 54 corresponds with the first stent component 14, the distal end 52 corresponds with the second stent component 12, and the arms 60 and 62 of the distal end 52 correspond with the arms 20 and 22 of the second stent component 12, as illustrated in FIGS. 4A-4C. As illustrated in FIG. 4B specifically, the rolled and collapsed second stent component 12 is bent in half at the junctional region 38 with its two arms 20 and 22 in parallel alignment with each other. The first stent component 14 is connected to the second stent component 12 at the pivot joint 34 at junction points 16. FIG. 4C illustrates the rolled and collapsed stent 10 inside the branched wire 50 prior to deployment or expansion.

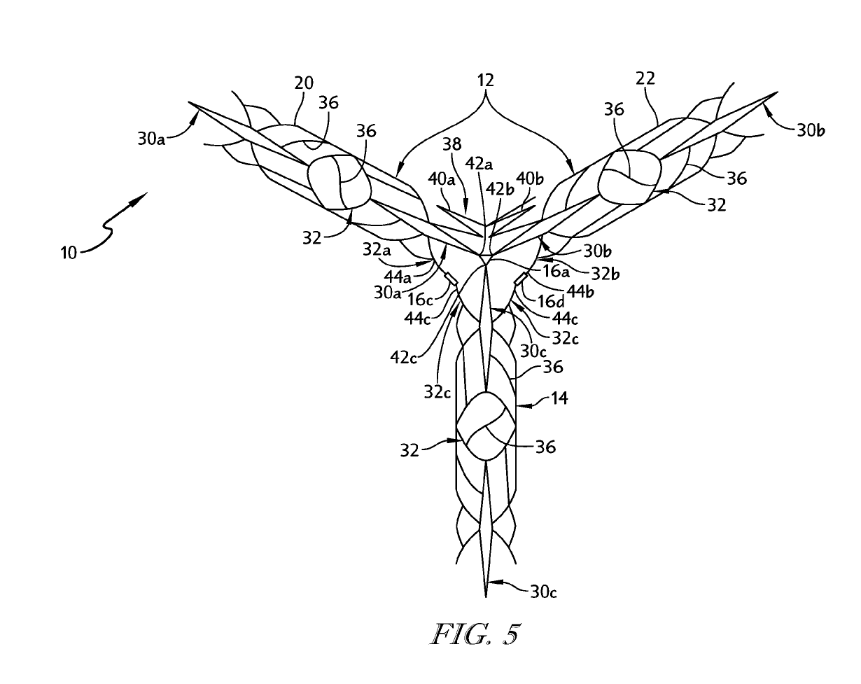

In illustrative embodiments, the stent 10 may be inserted into the vessel through the use of a catheter (not shown), as more fully described in WO 2013/009976. For example, the catheter may constrain the stent 10 as it is inserted into the vessel, and then the catheter may be removed or modified such that it no longer constrains the stent 10. When the stent 10 is no longer constrained, it naturally expands to an expanded configuration, as shown in FIG. 5.

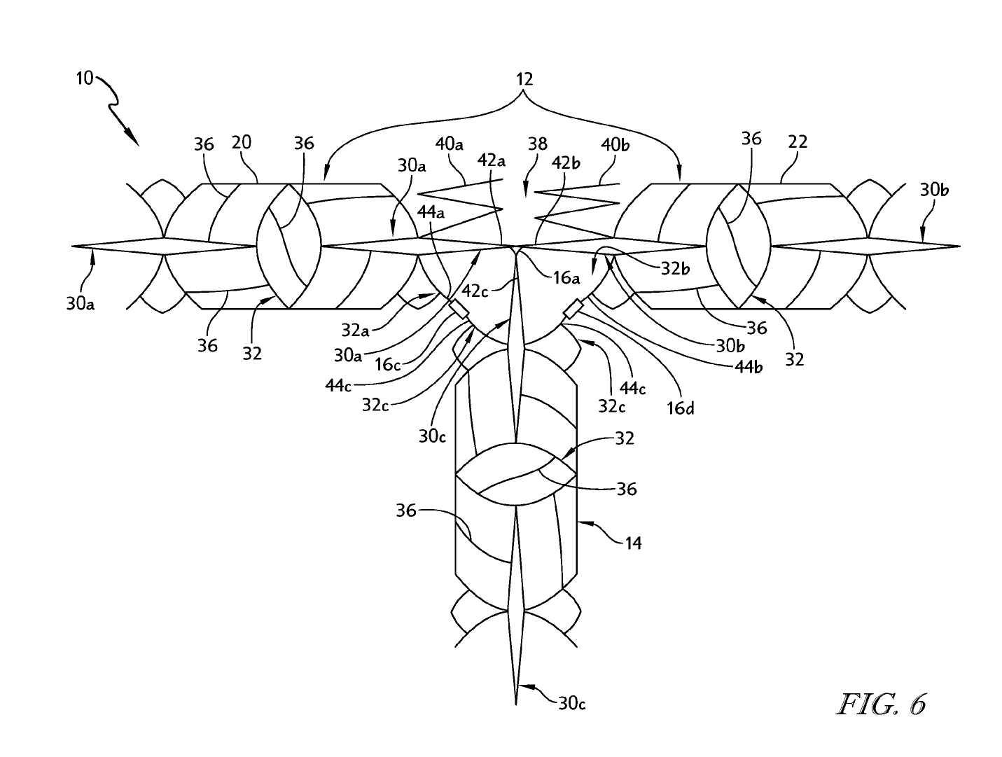

FIG. 5 and FIG. 6 show the complete stent 10 in a rolled and expanded configuration as assembled by coupling the first and second stent components 14 and 12 at the junction points 16. Specifically, FIG. 5 illustrates the rolled stent 10 after partial expansion and FIG. 6 illustrates the rolled stent 10 after full expansion. Four junction points, 16a, 16b, 16c, and 16d, connect the two stent components 14, 12 of the stent 10. Two of these junction points 16a and 16b connect the arms 20 and 22 of the second stent component 12 with the first component 14 by connecting the apex 42a of a longitudinally-oriented diamond 30a of the arm 20, the apex 42b of a longitudinally-oriented diamond 30b of the arm 22, and an apex 42c of a longitudinally-oriented diamond 30c of the first stent component 14. Because of the viewpoint, only junction point 16a is shown in FIGS. 5 and 6.

Another junction point 16c connects the arm 20 of the second stent component 12 with the first component 14 by connecting an apex 44a of an axially-oriented diamond 32a of the arm 20 and an apex 44c of an axially-oriented diamond 32c of the first stent component 14. The last junction point 16d connects the arm 22 of the second stent component 12 with the first component 14 by connecting an apex 44b of an axially-oriented diamond 32b of the arm 22 and an apex 44c of an axially-oriented diamond 32c of the first stent component 14. Similar to the joining of stent components 20,22 at junction 34 in FIG. 3, the four junction points 16 of the stent 10, can be connected by a variety of methods including, but not limited to, braided metal and/or plastics, a metal and/or plastic joining component, or other biocompatible joining mechanism.

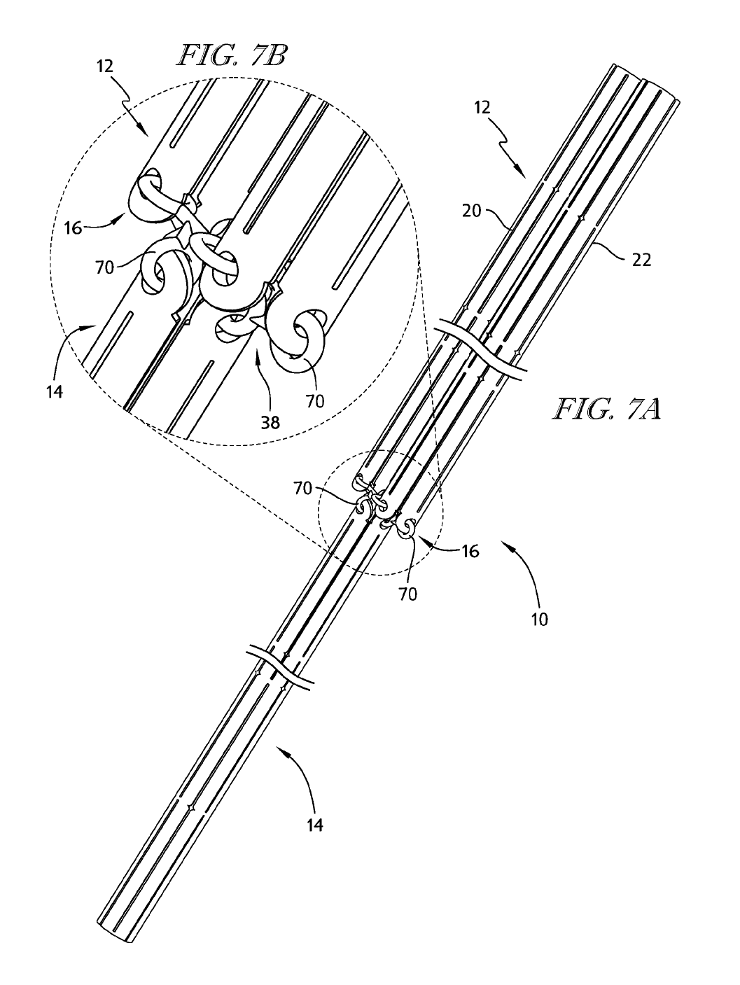

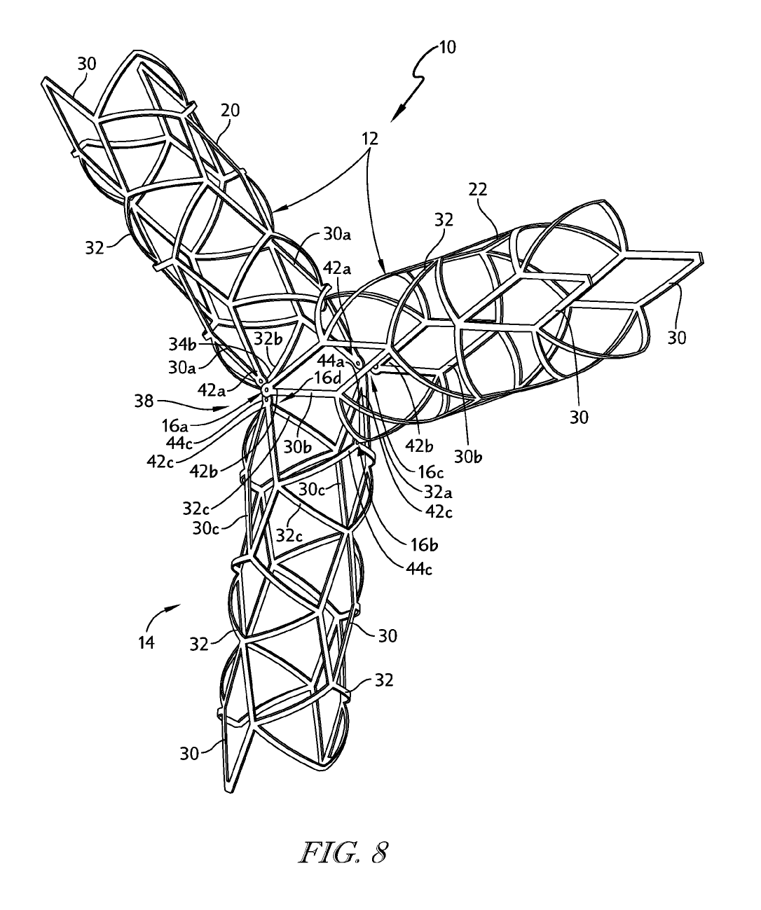

FIGS. 7A, 7B, and 8 show a further embodiment of a complete stent 10 as assembled by coupling the first and second stent components 14, 12 at the junction points 16a-16d. Specifically, FIGS. 7A and 7B illustrate the rolled and collapsed stent 10 before expansion and FIG. 8 illustrates the rolled and expanded stent 10 after full expansion. Four junction points 16a, 16b, 16c, 16d connect the two stent components 14, 12 of the stent. Two of these junction points 16a, 16c connect the arms 20, 22 of the second stent component 12 with the first component 14 by connecting the apex 42a one of the opposing longitudinally-oriented diamonds 30a of the arm 20, the apex 42b of one of the opposing longitudinally-oriented diamonds 30b of the arm 22, and the apex 42c of one of the opposing longitudinally-oriented diamonds 30c of the first stent component 14.

Another junction point 16b connects the arm 20 of the second stent component 12 with the first component 14 by connecting an apex 44a of an axially-oriented diamond 32a of the arm 20 and an apex 44c of an axially-oriented diamond 32c of the first stent component 14. The last junction point 16d connects the arm 22 of the second stent component 12 with the first component 14 by connecting the apex 44b of an axially-oriented diamond 32b of the arm 22 and an apex 44c of an axially-oriented diamond 32b of the first stent component 14. Similar to the joining of the stent components 20, 22 at junction 34 in FIG. 3, the four junction points 16a-16d of the stent 10 can be connected by a variety of methods including, but not limited to, braided metal and/or plastics, a metal and/or plastic component 70, as seen in FIGS. 7A and 7B, or any other suitable biocompatible joining mechanism.

Because a stent 10 once deployed/expanded will assume a precise and expected orientation within a blood vessel, the stent 10 can be modified to meet the needs of a particular patient's anatomy and pathology. The example stent 10 as described and shown in the FIGS. is merely a basic scaffold structure upon which an infinite number of additional stent features may be added or modified. Such features include, but are not limited to: variable porosity along the site of pathology or for protection of normal anatomy; matching of a bifurcation angle by changing the angle ratios of the longitudinally-oriented diamonds 30 or axially-oriented diamonds 32 of the stent 10; variation of size of the arms 20, 22 of the second stent component 12; and variation of diameter of the stent subunits 24.

In illustrative embodiments, in the open or expanded state, the stent 10 may be branched so that the distal arms 20 and 22 of the second stent component 12 form an angle with the first stent component 14, typically between 90 and 180 degrees. Thus, an angle between the two distal arms 20 and 22 may be between 0 to 180 degrees. This angle may be maintained by the bend of the second stent component 12 at the junctional region 38.

In illustrative embodiments, in the closed or collapsed state, the stent 10 may be configured to pass through a single catheter lumen (not shown) before reaching the end of the deployment catheter (not shown). In this collapsed state, the angle between the first stent component 14 and the arms 20 and 22 of the second stent component 12 must approximate 180 degrees. Thus, the arms 20 and 22 of the second stent component 12 must be bent so that they are parallel to each other during delivery through the catheter. The branched wire 50 may assist with delivery or deployment of the stent 10 in the catheter and/or blood vessel. Thus, unique features of the stent design allow the stent 10 to be delivered as a single unit, where the collapsed state is ideal for delivery, and the expanded state is ideal for final stent position, particularly in a bifurcated vessel.

Examples of use are provided herein for illustrative purposes, and are not intended to limit the scope of the disclosure. In one embodiment, the stent 10 may be used for the treatment of an intracranial aneurysm. The second stent component 12 may be deployed with or without the first stent component 14 to cover a neck of the intracranial aneurysm in order to aid in curative embolization. In another embodiment, the stent 10 may be used to treat stenosis of the vessels at a bifurcation. Bifurcations include, but are not limited to, those of the coronary arteries, carotid arteries, intracranial arteries, aortic bifurcation, and peripheral vessels.

What is claimed is:

1. A self-expanding stent with a collapsed configuration and an expanded configuration, comprising:
a first stent component including one or more longitudinally-oriented diamonds and one or more axially-oriented diamonds;
a second stent component including a first arm and a second arm, the first and second arm being coupled together at a pivot joint, the first and second arm including one or more longitudinally-oriented diamonds and one or more axially-oriented diamonds;
wherein the longitudinally-oriented diamonds are configured to substantially hold their shape in both the expanded configuration of the stent and the collapsed configuration of the stent and the axially-oriented diamonds are configured to expand from the collapsed configuration to the expanded configuration; and
wherein the first stent component is coupled to the second stent component adjacent the pivot joint.

2. The self-expanding stent of claim 1, wherein the pivot joint is created by coupling at least one apex of a longitudinally-oriented diamond of the first arm with at least one apex of a longitudinally-oriented diamond of the second arm.

3. The self-expanding stent of claim 1, wherein one or more stent scaffolding elements is located adjacent to the pivot joint of the second stent component, opposite the connection to the first stent component.

4. The self-expanding stent of claim 3, wherein the scaffolding element connects a space between the first arm and the second arm.

5. The self-expanding stent of claim 3, wherein the scaffolding element is coupled to the second stent component by biocompatible flexible and/or elastic joining material.

6. The self-expanding stent of claim 1, wherein one or more medium struts are coupled to and bisect the axially-oriented diamonds.

7. The self-expanding stent of claim 1, wherein the stent is configured to be placed at the junction of a bifurcated or branched blood vessel.

8. The self-expanding stent of claim 7, wherein the second stent component is configured to extend into the branched portion of the blood vessel, and wherein the first arm extends through a first blood vessel and the second arm extends through a second blood vessel to abut against walls of the blood vessels when the stent is the expanded configuration.

9. The self-expanding stent of claim 8, wherein the first arm extends at an angle between 0 and 180 degrees from the second arm when the stent is in the expanded configuration.

10. The self-expanding stent of claim 7, wherein the first stent component is configured to extend into a portion of the blood vessel prior to bifurcation.

11. The self-expanding stent of claim 10, wherein the wherein the second stent component is configured to extend into the branched portion of the blood vessel, and wherein the first arm extends through a first blood vessel and the second arm extends through a second blood vessel to abut against walls of the blood vessels when the stent is the expanded configuration.

12. The self-expanding stent of claim 11, wherein the angle between the first arm and the first stent component is between 90 and 180 degrees and the angle between the second arm and the first stent component is between 90 and 180 degrees.

13. The self-expanding stent of claim 1, wherein the first stent component and the second stent component are configured to be placed in a bifurcated or branched blood vessel at the same time.

14. The self-expanding stent of claim 1, wherein the second stent component is configured to be folded or bent about the pivot joint in the collapsed configuration.

15. The self-expanding stent of claim 14, wherein the first and second arms are configured to be in parallel alignment with the first stent component when in the collapsed configuration.

16. The self-expanding stent of claim 15, wherein the angle between the first and second arms expand to a predetermined angle between 90 and 180 degrees when in the expanded configuration.

17. The self-expanding stent of claim 15, wherein the first and second arms are configured to be at an angle between 90 and 180 degrees with respect to the first stent component when in the expanded configuration.

18. The self-expanding stent of claim 1, wherein the stent is created in a manner customized to the targeted delivery location in a blood vessel.

19. The self-expanding stent of claim 18, wherein the stent comprises a shape memory material.

20. The self-expanding stent of claim 18, wherein the stent is formed using three-dimensional printing

21. The self-expanding stent of claim 1, wherein the first and second stent components are constructed as a solitary unit.

22. The self-expanding stent of claim 1, wherein the stent is created in a manner customized to the targeted location for delivery, using a shape memory material and/or three-dimensional printing.

23. A method of making a bifurcated self-expanding stent with a collapsed configuration and an expanded configuration, comprising:
creating a first stent component with one or more longitudinally-oriented diamonds and one or more axially-oriented diamonds;
creating a second stent component with a first arm and a second arm, the first and second arm including one or more longitudinally-oriented diamonds and one or more axially-oriented diamonds;
coupling the first and second arm being together at a pivot joint; and
coupling the first stent component to the second stent component adjacent the pivot joint.

24. A method of deploying a bifurcating self-expanding stent in a bifurcated vessel of a vascular system, comprising:
locating the bifurcated vessel in the vascular system, the bifurcated vessel including a first branching section, a second, distal branching section and a third, distal branching section that join together at a junction of the bifurcated vessel;
providing a bifurcated catheter into the vascular system, the bifurcated catheter including a proximal first leg connected to a distal second leg and a distal third leg at a junction of the bifurcated catheter, the bifurcated catheter including a splitable seam that extends along the second and third legs of the bifurcated catheter;
providing the bifurcated stent with a first stent component and a second stent component, the first and second stent components including one or more longitudinally-oriented diamonds and one or more axially-oriented diamonds, the second stent component further including a first arm and a second arm coupled together at a pivot joint, and wherein the first stent component is coupled to the second stent component adjacent the pivot joint;
inserting the bifurcated stent into the bifurcated catheter wherein the first arm of the bifurcated stent is disposed in the distal second leg of the catheter, the second arm of the bifurcated stent is disposed in the distal third leg of the catheter, and the first stent component of the bifurcated stent is disposed in the first leg of the bifurcated catheter;
sequentially inserting the bifurcated catheter and bifurcated stent into the bifurcated vessel, positioning the first leg of the bifurcated catheter and first stent component in the first branching section of the vessel, positioning the second leg of the bifurcated catheter and the first arm of the bifurcated stent in the second, distal branching section of the vessel, and positioning the third leg of the bifurcated catheter and the second arm of the bifurcate stent in the third, distal branching section of the vessel;
splitting the seam of the bifurcated catheter;
withdrawing the bifurcated catheter; and
allowing the bifurcated stent to expand against interior surfaces of the first branching section, second, distal branching section and third, distal branching section of the bifurcated vessel.
